(19) United States
(12) Patent Application Publication
(10) Pub. No.: US 2009/0247454 A1
(43) Pub. Date: Oct. 1, 2009

(54) Method for detecting a predisposition to develop gestational diabetes mellitus and treatment for this disease

(75) Inventors: Jose Manuel Fernandez-Real Lemos, Girona (ES); Wifredo Ricart Engel, Girona (ES)

Correspondence Address: BERENBAUM, WEINSHIENK & EASON, P.C, 370 17TH STREET, SUITE 4800, DENVER, CO, 80202, US

(73) Assignee: MELLITUS, S.L., Barcelona (ES)

(21) Appl. No.: 11/576,339

(22) PCT Filed: Sep. 27, 2005
(86) PCT No.: PCT/EP05/54838

§ 371 Date: Mar. 29, 2007

(30) Foreign Application Priority Data
Sep. 29, 2004 (EP) 04104777.0

(51) Int. Cl. A61K 38/17; C12Q 1/68; A61P 3/10

(52) U.S. Cl. 514/12; 435/6

(57) ABSTRACT

Methods and kits for detecting a predisposition to develop gestational diabetes mellitus and pharmacological treatment for this disease.

Described herein are methods and kits for detecting a predisposition to develop gestational diabetes mellitus and pharmacological treatment for this disease.

### BACKGROUND

Gestational diabetes mellitus (GDM) is a disease defined as a glucose intolerance of variable degree, which usually develops in the second and third trimester of pregnancy.

A wide range of complications are associated with GDM. For the mother, GDM increases the risk of preeclampsia, cesarean delivery, and future type 2 diabetes. In the fetus or neonate, the disorder is associated with higher rates of perinatal mortality, macrosomia, birth trauma, hyperbilirubinemia, and neonatal hypoglycemia

Initial screening for GDM is currently accomplished by performing a 50-g, one-hour glucose challenge test at 24 to 28 weeks of gestation. Serum or plasma glucose values of 130 mg per dL or more are generally considered abnormal. An abnormal one-hour screening test should be followed by a 100-g, three-hour venous serum or plasma glucose tolerance test. GDM is diagnosed when a patient has two or more abnormal values on a fasting 100-g, three-hour glucose tolerance test.

Despite decades of research, there is a lack of international consensus on the screening, diagnosis, treatment and follow up of GDM. Currently, GDM is managed by diet modification, exercise and exogenous human insulin. During the last decade, several insulin analogues and oral antihyperglycaemic drugs have been used in the management of diabetes in non-pregnant women. However, most of them remain in the investigational stage in GDM due to concerns about their safety in pregnancy (cf. M M Agarwal, et al., J. Expert Opin Investig Drugs. 2004, vol. 13(9), pp. 1103-11).

It is of paramount importance to identify women at risk of GDM, in order to avoid the consequences and complications related to this disease. New pharmacological treatment that could potentially benefit women with GDM is also desirable.

Mannan-binding lectin (MBL) is a plasma protein synthesized in the liver and released as a component of the acute-phase response (cf. R Medzhitov and C. Janeway, Innate Inmunity. N Eng J Med. 2000, vol. 343, pp. 338-344,). It is a member of the collectin family of proteins and is considered an important component of the innate immune system. MBL binds to an array of specific repetitive carbohydrate structures on microbial surfaces and subsequently exerts an antibacterial effect by the activation of the complement cascade through MBL-associated serine proteases, the so-called MBL pathway, or by promoting phagocytosis. MBL levels are genetically determined, although a large interindividual variability exists, in part due to its behaviour as a reactant phase protein. For this reason, genetic studies have recently gained interest. Three major mutant alleles in exon 1, as well as mutations in the promoter region of the gene have been associated with MBL deficiency. The presence of MBL deficiency in 10% of the population makes it the most frequent immunodeficiency described. Recurrent infections (cf. J A Summerfield, et al. BMJ. 1997, vol. 314, pp. 1229-nn), recurrent miscarriage (cf. D C Kilpatrick, et al. Human reproduction. 1999, vol. 14, pp. 2379-2380) and a greater risk of having autoimmune disorders such as systemic lupus erythematosus (cf. J Villarreal, et al. Rheumatology. 2001, vol. 40, pp. 1009-12), rheumatoid arthritis (cf. N A Graudal, et al. J Rheumatol. 1998, vol. 25, pp. 629-35) and perhaps type 1 diabetes mellitus (cf. A Tsutsumi, et al. Human Immunology. 2003, vol. 64, pp. 621-624) have been related with MBL deficiency.

Several patents and patent applications describe the use of MBL for treating disorders mainly associated with infections. Thus, EP1181038B1 discloses the use of mannan-binding lectin sub-unit or oligomer compositions, for the prophylaxis or treatment of infections, particularly in individuals having an immunocompromized condition; WO04026330A1 discloses the use of blood mannan-binding lectin regulator in manufacture of a medicament to treat critically ill patients having multiple organ failure, post-surgical critical illness or post-traumatic critical illness; US20040029785A1 discloses the use of a composition comprising a mannan-binding lectin (MBL) sub-unit for manufacturing an infection medicament for use in an individual being treated with a tumor necrosis factor (TNF)-alpha inhibitor; WO0070043A1 discloses the production of a human recombinant mannan binding lectin composition for treating disorders associated with chemotherapy, HIV, by transforming host cell culture with gene expression construct and cultivating culture.

WO0222161A2 discloses the treatment or prophylaxis of diseases associated with disturbances in the complement/lipid pathway, in particular atherosclerosis, by modulating the activity of one or more elements of said pathway, e.g., mannose-binding lectins, C4A, C4B, C2, vitronectin, clusterin.

### SUMMARY

Described herein is a method for detecting an increased susceptibility to developing gestational diabetes mellitus (GDM) and providing a potential pharmacological treatment for this disease.

Also described herein are the effects of polymorphisms in MBL2 gene in a group of 105 consecutive GDM women and 173 healthy pregnant women (see example 1). Surprisingly, based on the results obtained, it has been found that mutations for MBL in a European population (preferably the most frequent mutation G54D in exon 1 of MBL2 gene) are associated with an increased susceptibility to developing GDM. These results are also confirmed by the lower mean MBL plasmatic levels in the group of women carrying the mutant alleles.

The results obtained would be in agreement with the line of evidence that insulin resistance is the result of a low-grade chronic inflammatory state, where any situation that maintains or perpetuates an inflammatory response will favour diminished insulin sensitivity. A major effector function of MBL is the activation of complement, a factor known to influence the inflammatory response (cf. P J Lehner, et al. Lancet. 1992, vol. 340, pp. 1379-81). In addition, MBL has the ability to enhance phagocytosis (cf. A J Tenner, et al. Immunity. 1995, vol. 3, pp. 485-493) and to inhibit TNF-α release (cf. M. Soell, et al. J Immunol. 1995, vol. 154, pp. 851-860). This cytokine has been repeatedly and strongly associated with the degree of insulin resistance. According to these observations, and as a possible explanation for the results obtained, women carrying the mutant MBL haplotype, due to decreased MBL levels, may have a modified activation of the innate immunity response in front of an aggression and make the mother and foetus more susceptible to a prolonged and sustained inflammatory response. Consequently, there may be a sustained release of inflammatory cytokines known to down-regulate the insulin sensitivity, such as TNF-alpha, and this situation, in part, could contribute to the appearance of GDM.

Accordingly, a first aspect herein relates to a kit for detecting a predisposition to gestational diabetes mellitus (GDM) in a woman, with an appropriate means for detecting, in a sample taken from said woman, the presence or the absence of:
(i) at least one mutation, in exon 1 of the MBL2 gene, selected from the group consisting of a mutation that provokes the amino acid substitution R52C, a mutation that provokes the amino acid substitution G54D and a mutation that provokes the amino acid substitution G57E in the corresponding encoded polypeptide; and/or
(ii) the haplotype HY in the promoter region of the MBL2 gene.

This first aspect may alternatively be formulated as a method of detecting a predisposition to GDM in a woman, by detection of the presence or the absence of the mutations or haplotype as described above.

A second aspect relates to the use of a composition with at least one mannan-binding lectin (MBL) subunit, or at least one mannan-binding lectin (MBL) oligomer with at least one mannan-binding lectin (MBL) subunit for the manufacture of a medicament for the preventive or therapeutic treatment of gestational diabetes mellitus (GDM) in a woman who one or the other or both:
(i) carries at least one mutation, in exon 1 of the MBL2 gene, selected from the group consisting of a mutation that provokes the amino acid substitution R52C, a mutation that provokes the amino acid substitution G54D and a mutation that provokes the amino acid substitution G57E in the corresponding encoded polypeptide; and/or
(ii) does not carry the haplotype HY in the promoter region of the MBL2 gene.

This second aspect may alternatively be formulated as a method for the preventive or therapeutic treatment of GDM by administering to a pregnant woman, who carries at least one mutation and/or does not carry the haplotype as described above, an effective amount of a composition with at least one mannan-binding lectin (MBL) subunit, or at least one mannan-binding lectin (MBL) oligomer comprising at least two mannan-binding lectin (MBL) subunits.

The method of prognosing of GDM based on the presence of a mutation or absence of the haplotype as described above may allow detection of the susceptibility to developing GDM with more anticipation than the current detection methods, before it develops. Therefore it may allow implementing preventive measures prior to the onset of the disease.

Using MBL for the treatment or prophylaxis of GDM, due to its endogenous production, would raise fewer concerns about safety in pregnancy than the several insulin analogues and oral antihyperglycaemic drugs that have been used in the management of diabetes in non-pregnant women and that are still in investigational phase for GDM.

### DETAILED DESCRIPTION

### MBL2 Genotype

The gene codifying for human MBL, MBL2 gene, is located on chromosome 10 at q11.2-q21. Three mutations are known in the structural region of the molecule (codons 52, 54 and 57) giving rise to three allelic variants called D, B and C, respectively, while the wild type is called A. The three point mutations occur at nucleotides 223 (C to T), 230 (G to A) and 239 (G to A) of exon 1 for the D, B and C alleles, respectively. This causes the substitution of arginine by cysteine at codon 52 (R52C), the substitution of glycine by aspartic acid at codon 54 (G54D) and the substitution of glycine by glutamic acid at codon 57 (G57E). These amino acid substitutions are thought to affect the tertiary structure of the collagenous region of the protein. Additional polymorphisms are found in the promoter region of the gene. Two promoter variants, H and L at position 550 are in linkage disequilibrium with the X and the Y variant at position 221 and are found as three haplotypes e.g. HY, LY and LX. The HY is associated with the highest plasma levels of MBL, the LY haplotype with intermediate levels and the LX haplotype is associated with the lowest circulating plasma levels of MBL. Due to the linkage disequilibrium, only seven haplotypes (HYPA, LYQA, LYPA, LXPA, LYPB, LYQC and HYPD) are commonly found (cf. H. O. Madsen, et al., J. Immunol. 1995, vol. 155, pp. 3013).

In this description, positions on the MBL2 gene and on the corresponding encoded polypeptide, are given with reference to the GenBank Accession Number NM_{—}000242. This reference corresponds to the Homo sapiens mannose-binding lectin (protein C) 2, soluble (opsonic defect) (MBL2), mRNA.

### Detection of the GDM Predisposition

The kit described herein may be used to detect a predisposition to GDM in a woman. The presence of one or more of the mutations and/or the absence of the haplotype as described above, in heterozygous or homozygous forms may be associated with an increased predisposition to develop GDM.

The kit may detect the presence or the absence of the G54D mutation; accordingly, the kit may have appropriate means for detecting the presence or the absence of a mutation, in exon 1 of the MBL2 gene that provokes the amino acid substitution G54D in the corresponding encoded polypeptide.

The term predisposition, as used herein, is to be understood broadly, as the quality or state of being susceptible, the state of being predisposed to, sensitive to, or of lacking the ability to resist something (as a pathogen, familial disease, etc), or having an increased risk of developing a disease.

The kit may include means based on genotyping techniques well-known to those skilled in the art. These techniques should be able to read completely or partially (e.g. the G54D mutation region, but also the remaining mutations) MBL2 genotype or to distinguish selectively e.g. G54D from the other mutations on MBL2 gene. The presence of the polymorphism may be detected using one or more oligonucleotides which hybridize to a nucleotide sequence comprising the polymorphism on the MBL2 gene. In this description, hybridization to an MBL2 gene when double-strand may include hybridization to one strand or to the complementary thereof. Oligonucleotides may be fluorescently, chemiluminescently or radioactively labelled to act as probes and detect the nucleotide sequence comprising the polymorphism. These probes may be used, for example, in microarrays on glass support or in bead-based microarrays.

The kit may include PCR technology. Examples of PCR-based methods include restriction fragment length polymorphism (RFLP), site-directed mutagenesis (SDM), sequence-specific oligonucleotide (SSO) hybridization, nested primer, DNA heteroduplexes, or amplification refractory mutation system-PCR (ARMS-PCR).

The art describes suitable PCR based technologies to specifically detect mutations on MBL2 gene. An example is the use of PCR with sequence-specific primers (PCR-SSP) (cf. R. Steffensen et al., Journal of Immunological Methods 2000, vol. 241, pp. 33-42).

Real-time PCR with fluorescent hybridisation probes may also be used for genotyping MBL2 gene mutations (cf. R. Steffensen et al., Journal of Immunological Methods 2003, vol 278, pp. 191-9). Real-time PCR may monitor the fluorescence emitted during the reaction as an indicator of amplimer production during each PCR cycle (i.e., in real time), as opposed to the endpoint detection by conventional quantitative PCR methods. The real-time PCR system may be based on the detection and quantification of a fluorescent probe acting as a reporter. In this approach, PCR and melting temperature (Tm) curve analysis may be combined based on the principle of mutation detection by melting point analysis with fluorescence resonance energy transfer (FRET) hybridisation probe.

The art also describes improvements on MBL2 genotyping based on real-time PCR methods. One approach may be based on the 5′ nuclease (TaqMan) assay in combination with the use of minor-groove-binder (MGB) probes. In contrast to conventional probes, MGB probes have a short length and can be used for detection of mutations that are in close proximity to each other, as is the case for the structural mutations in exon 1 of the MBL gene (cf. E. Van Hoeyveld et al., Journal of Immunological Methods 2004, vol. 287, pp. 227-30).

The kit may also contain appropriate instructions for carrying out the detection. Instructions may include those rules on how to make use of the reagents and instrumentation suitable to carry out the detection of the presence or the absence of the mutation in the MBL2 gene. Furthermore, instructions may also include those rules on how to interpret the results and link the results with the predisposition to suffer from GDM. An example of this may be defined fluorescence patterns for comparing the detected signals in real time PCR.

Accordingly, the kit may have appropriate instructions explaining that:
(i) the presence of at least one mutation, in exon 1 of the MBL2 gene, selected from the group consisting of a mutation that provokes the amino acid substitution R52C, a mutation that provokes the amino acid substitution G54D and a mutation that provokes the amino acid substitution G57E in the corresponding encoded polypeptide; and/or
(ii) the absence of the haplotype HY in the promoter region of the MBL2 gene, results in an increased predisposition to have or develop gestational diabetes mellitus (GDM).

These instructions may be related to the G54D mutation.

The detection may be performed with a separated sample from the woman susceptible to GDM. This woman may be in the first trimester of pregnancy or willing to get pregnant. The sample may be a tissue or a fluid, generally blood, taken from the individual. Depending on the selected technique, the sample may be processed to obtain isolated cells, protein fraction or nucleic acids fraction (e.g. genomic DNA or messenger RNA).

### MBL Protein

Mannan-binding lectin (MBL) is also known as mannose-binding lectin, mannan-binding protein or mannose-binding protein (MBP).

The polypeptide chain of secreted MBL is 228 amino acids long (not including the 20-residue signal peptide), and consists of a 20-residue ‘cysteine-rich’ region (containing 3 cysteines), followed by a collagenous region containing 19 Gly-Xaa1-Xaa2 triplets, a ‘neck’ region, and then a C-terminal calcium-dependent carbohydrate-binding lectin domain, also called a carbohydrate-recognition domain (CRD). The neck region forms an alpha-helical coiled-coil structure which possibly promotes trimerization of three polypeptides to form the subunit. The trimer is stabilized by hydrophobic interactions and inter-chain disulphide bonds within the N-terminal cysteine-rich region. MBL subunits assemble into larger oligomeric structures forming a ‘bunch-of-tulips’ or sertiform appearance. The most common oligomeric form in humans appear to be a six-subunit (18-polypeptide chain) form with an overall molecular mass of about 18×25000 Da. Unusually, disulphide bridging within and between subunits is incomplete and variable, so that, for example, the common six-subunit form is heterogeneous, consisting of a number of isoforms with different disulphide bridging. This may be evident from SDS/PAGE analysis of non-reduced MBL, and comparison with analyses made by non-denaturing hydrodynamic methods. Several publications suggest that native MBL in serum or plasma may occur in oligomeric forms of different sizes (ranging from one to six subunits in humans), but it appears likely that the six-subunit oligomer is by far the major form; smaller oligomers may form on storage or processing of plasma. (cf. J. S. Presanis, et al., Biochem. Soc. Trans. 2003, vol. 31, pp. 748-752).

The repeating sugar structures on microbial surfaces, not generally found on mammalian surfaces, can bind with high avidity to the CRDs which, within the trimeric head of each subunit, are spaced 45-55 Å apart. The specificity of MBL in recognizing sugar patterns on surfaces (pathogen-associated molecular patterns or PAMPs) may rely on the identity of the monosaccharide, its exposure on the surface and spacing between sugar residues. The spacing between the individual CRDs within one subunit, and the spacing of subunit heads relative to each other, are the determining factors (cf. J. S. Presanis, et al., Biochem. Soc. Trans. 2003, vol. 31, pp. 748-752).

### Suitable MBL

The MBL composition used to manufacture an MBL medicament, may contain at least one mannan-binding lectin (MBL) subunit, or at least one mannan-binding lectin (MBL) oligomer with at least two mannan-binding lectin (MBL) subunits.

The composition may have at least one mannan-binding lectin (MBL) oligomer with at least two mannan-binding lectin (MBL) subunits. The oligomer may be selected from the group of oligomers consisting of tetramers, pentamers and hexamers. In an embodiment said oligomer is a hexamer.

### Administration of MBL

The MBL composition may be administered to a woman who:
(i) carries at least one mutation, in exon 1 of the MBL2 gene, selected from the group of a mutation that provokes the amino acid substitution R52C, a mutation that provokes the amino acid substitution G54D and a mutation that provokes the amino acid substitution G57E in the corresponding encoded polypeptide; and/or
(ii) does not carry the haplotype HY in the promoter region of the MBL2 gene.

The woman may carry a mutation in the MBL2 gene provoking the amino acid substitution G54D in the corresponding encoded polypeptide. The pregnant woman carrying the mutation may be heterozygote (GA) or homozygote (AA) for the mutation.

The purpose of the administration of an MBL composition may be preventive (to avoid the development of these diseases) and/or therapeutic (to treat these diseases once they have developed.

The treatment may be therapeutic and administered to a pregnant woman that suffers from GDM.

The treatment may be preventive and administered to a woman who is pregnant or is willing to get pregnant. Such a woman may not necessarily suffer GDM at the moment, but may have suffered from GDM in a previous pregnancy.

MBL oligomers may be administered at doses of about 6 mg, about 2 or 3 times per week and the amount may be administered in divided doses on a weekly basis. The particular dose may be varied within or without the range that is specified herein depending on the particular application or severity of a disease. Those who are skilled in the art may ascertain the proper dose using standard procedures. The dose may be an effective amount of MBL oligomers in the sense that improved insulin resistance is seen in the treated subject. A suitable assay for testing improved insulin resistance is known to one skilled in the art and guidance may be found in the working examples herein.

### Compositions

The medicament may be in the form of a pharmaceutical composition. Such a medicament/pharmaceutical composition may be administered by any means that achieves their intended purpose. For example, administration may be by parenteral routes, including subcutaneous, intravenous, intradermal, intramuscular, intraperitoneal, intrathecal, transdermal, or buccal routes. Alternatively, or concurrently, administration may be by the oral or rectal route. The administration may also be pulmonal or topical. The pharmaceutical compositions may be administered parenterally by bolus injection or by gradual release over time.

The composition may be administered by depot administration once a month.

In addition to the MBL polypeptide oligomers, the medicament may comprise a pharmaceutically acceptable carrier substance and/or vehicles. In particular, a stabilising agent may be added to stabilise the MBL protein. The stabilising agent may be a sugar alcohol, saccharides, proteins and/or amino acids. An example of a stabilising agent may be albumin.

Other conventional additives may be added to the medicament depending on administration form. The medicament may be in a form suitable for injections. Conventional carrier substances, such as isotonic saline, may be used.

The medicament may also be in a form suitable for pulmonal administration, such as in the form of a powder for inhalation or cream or fluid for topical application.

The MBL composition may also be administered as a depot (e.g. an injectable depot composition) to the patient. The depot may be made with an adequate release profile of MBL and the patient may thereby, in a comfortable easy way, get a continued improved insulin resistance and thereby continued normalized blood glucose levels.

Accordingly, the medicament may contain a depot composition, such as an injectable depot composition. After introduction into the patient (animal or human), the depot may preferably have an adequate release profile of MBL. Numerous suitable depot compositions are known in the art and may be made with various adequate release profiles. See e.g. U.S. Pat. No. 6,331,311, “Injectable depot gel composition and method of preparing the composition” for further details with respect to suitable depot compositions. The injectable depot composition is an injectable depot gel composition.

The depot composition may be administered by implanting, possibly subcutaneously, a suitable device. An example of such a suitable device is a so-called pump, such as the commercially available ALZET® Osmotic Pump from the company DURECT Corporation, USA. Suitable adequate pumps are known to those skilled in the art and they provide the possibility of providing continuous delivery (i.e. by subcutaneously implantation), thereby eliminating the need for frequent, round-the-clock injections. Adequate release profiles may be a release profile allowing the depot to be administered in a period interval from about 1 to about 3 months.

Preferably, the preparations contain from about 0.1 to about 99 percent, preferably from about 25-85 percent, of active compound(s), together with the excipient.

### Source of MBL

The MBL composition used to manufacture an MBL medicament may be produced from any MBL source available.

The MBL source may be natural MBL, whereby the MBL polypeptides are produced in a native host organism, meaning that MBL is produced by a cell normally expressing MBL. One method of producing an MBL composition is by extraction of MBL from human body liquids, such as serum or plasma.

The MBL may be of human origin.

The MBL source may be serum, from which an MBL composition may be obtained by purifying serum, plasma, milk product, colostrum or the like by a suitable purification method, such as affinity chromatography using carbohydrate derivatised matrices, such as mannose or mannan matrices. Such a method is discussed in WO99/64453.

The MBL composition used to manufacture an MBL medicament may have MBL oligomers having a size distribution substantially identical to the size distribution of MBL in serum, such as a size distribution profile at least about 80% identical to the size distribution profile of MBL in serum, a size distribution profile at least about 90% identical to the size distribution profile of MBL in serum, a size distribution profile at least about 95% identical to the size distribution profile of MBL in serum.

The matrix for the purification of MBL may be derivatized with any carbohydrate or carbohydrate mixture to which MBL binds. The matrix is preferably a mannose-, a fucose, a N-acetylglucosamin or a glucose derivatized matrix, such as most preferably a mannose matrix.

The selectivity of the carbohydrate-derivatized matrix may be obtained by ensuring that the matrix as such, i.e. the un-derivatized matrix has substantially no affinity to MBL polypeptides. This may occur when the matrix as such is carbohydrate-free. The matrix may be in any form suitable for the chromatography, mostly in the form of beads, such as plastic beads. A purification method is described in international patent application WO0070043 and WO9937676.

The MBL polypeptide oligomers may also be produced by a host organism not natively expressing an MBL polypeptide, such as by recombinant technology.

A clinical grade MBL composition may be obtained by using an MBL source produced by recombinant technology, wherein the MBL source is the culture media from culturing MBL-producing cells.

Described herein is MBL produced by a process of producing a human recombinant mannan binding lectin (MBL) polypeptide, made by
preparing a gene expression construct comprising a DNA sequence encoding a human MBL polypeptide or a functional equivalent thereof,
transforming a host cell culture with the construct,
cultivating the host cell culture, thereby obtaining expression and secretion of the polypeptide into the culture medium, followed by
obtaining a culture medium of human recombinant MBL polypeptides.

The culture medium of human recombinant MBL polypeptides may then be purified as described above.

The gene expression construct may be produced by conventional methods known to one skilled in the art, such as described in U.S. Pat. No. 5,270,199.

In another embodiment the gene expression construct may be prepared as described in Danish Patent application No: PA 1999 00668 or in international patent application (WO0070043) having the title “Recombinant Human Mannan Binding Lectin”.

The expression may be preferably carried out in mammalian cells, which preparation results from the use of an expression vector with an intron sequence(s) from an MBL gene and at least one exon sequence. Transgenic animals as the expression system may be animals that have been genetically modified to contain and express the human MBL gene or fragments or mimics thereof. In addition to the purification method, it may be that the gene expression construct and the host cell also favour production of higher oligomers.

### EXAMPLES

### Example 1

### Relationship between MBL Polymorphism and Occurrence of GDM

### Introduction

The study detailed below investigated the hypothesis that a genetic predisposition to a proinflammatory state could favour the appearance of GDM during pregnancy.

To evaluate this question, plasma MBL levels and MBL polymorphisms were studied in pregnancy, a reversible insulin resistance state. Two groups of women were considered, one with normal glucose tolerance and the other with GDM.

### Materials and Methods

Patient population:

Between January 1999 and February 2001, 105 pregnant women with GDM were studied. They were compared with 173 pregnant women matched for geographic origin, parity and body mass index (BMI), (Table 1). The National Data Group criteria were used to define GDM (cf. NA Graudal, et al. J Rheumatol. 1998, vol. 25, pp. 629-35). All women had been followed-up at the diabetes clinic and the obstetric service of University Hospital “Joan XXIII” from Tarragona (Northeast of Spain). During pregnancy in the GDM group, 57.3% women were treated only with diet and 42.7 % were treated with diet plus insulin. All GDM women entered the same out patient diabetes education program, with the same team (physician and nurse practitioner) the gestation period, programming an individualized dietary plan (calculating a diet with 45% of carbohydrates) and 1 h postprandial blood glucose monitoring. When postprandial glucose response exceeded two fold 6.7 mmol/l, insulin therapy was started (cf. A Tsutsumi, et al. Human Immunology. 2003, vol. 64, pp. 621-624). The control group included 173 healthy women with a normal O'Sullivan test during pregnancy. All women were healthy (except for GDM) and were not taking any medication at the time of the study. Each subject gave informed consent before entering the study. The Ethical Committee of University Hospital “Joan XXIII” approved the study.

### Laboratory Measurements:

Venous blood sample was drawn after overnight fasting between weeks 24 and 28 (a), after an O'Sullivan Test was performed.

Plasma glucose was measured with a glucose oxidase method using a Hitachi autoanalyzer. Plasma levels of sTNFR1 and sTNFR2 were determined by a solid-phase enzyme-amplified sensitivity immunoassay (EASIA) performed on a micro titer plate (Medgenix sTNFR1-EASIA, sTNFR2-EASIA, BioSource Europe, Fleurus, Belgium). Intra and interassay coefficients of variation were <7 and <9%, respectively. The sTNFR2 EASIA does not cross-react with sTNFR1 and vice versa. TNF-α does not interfere with the assay.

Plasma levels of MBL were determined using commercially available MBL ELISA kits (AntibodyShop, Copenhagen, Denmark). The lower detection limit was 5 ng/mL for undiluted samples.

Plasma leptin concentrations were measured by radio immunoassay (Linco™ Research Inc. St. Charles, Mo., USA). The lower detection limit was 0.5 μg/L. Intra and interassay coefficients of variation were <7% and <8%, respectively. The radioimmunoassay for leptin did not exhibit cross-reactivity with human proinsulin, insulin or glucagon.

### DNA and PCR Methodology

DNA was extracted from EDTA blood samples using MasterPure™ Genomic DNA Purification Kit (Epicentre, Madison, USA).

The R52C and G54D polymorphisms located in exon 1 of MBL2 were analyzed by sequence analysis. A total of 100 ng genomic DNA was amplified with specific primers derived from the published sequence (GenBank: NM_{—}000242): 5′TCACTCCCTCTCCTTCTCCT3′ and 5′GTTCCCCCTTTTCTCCCTTG′. All PCR amplifications were carried out on a final volume of 25 μl containing 1× Buffer (10 mM Tris-HCl pH 8.4, 50 mM KCl), 1 mM MgCl₂, 0.2 mM of each dNTP, 0.2 μM of each primer, 1U Taq polymerase (GeneCraft, Germany). Amplification conditions consisted of initial denaturation at 94° C. for 3 min, followed by 35 cycles of denaturation at 94° C. for 30 s, annealing at 63.1° C. for 30 s, and extension at 72° C. for 30 s. The PCR profile ended with a final extension at 72° C. for 10 min. PCR reactions were carried out using a GeneAmp PCR system 9700 (PE Biosystems). The resulting 169 bp fragment was purified using High Pure PCR Product Purification Kit (ROCHE, Mannheim, Germany) Fluorescent-based automated sequencing of amplified product was performed on an ABI PRISM™ 310 Genetic Analyzer (Applied Biosystems) using dye-terminator methodology (BigDye™ Terminator v3.0, Applied Biosystems) according to the manufacturer's instructions.

### Statistical Analysis:

All statistical analysis was performed by using the SPSS/PC+ statistical package (v. 10.0 for Windows; Chicago, Ill., USA). Descriptive data are expressed as mean value ±SD. Differences in levels between groups were compared by using a Student's t test, or analysis of variance of clinical or laboratory parameters. Non-parametric test were performed when variables did not have a gaussian distribution. Logistic regression analysis was used to identify determinants of GDM, and ORs are presented with 95% CI, for significant ORs.

Multiple linear regression analysis was also used to analyse the independence of the association between quantitative variables.

### Results:

TABLE 1
Clinical and biochemical characteristics
of the healthy pregnant and GDM group.

| | Healthy pregnant | GDM |
|---|---|---|
| N | 173 | 103 |
| Age (years) | 29.7 ± 5.0 | 33.4 ± 4.4‡ |
| BMI (Kg/m²) | 24.4 ± 5.1 | 25.7 ± 5.3 |
| SBP (mm Hg) | 121.5 ± 13.6 | 117.2 ± 12.8* |
| DBP (mm Hg) | 76.2 ± 11.7 | 68.7 ± 10.4‡ |
| Parity | 0.96 ± 1.01 | 1.06 ± 1.06 |
| Increase in body weight (kg) | 12.5 ± 10.1 | 8.2 ± 4.6† |
| Fetal weight (g) | 3237.7 ± 547.7 | 3230.7 ± 482.7 |
| Time of delivery (weeks) | 39.02 ± 1.96 | 39.04 ± 1.66 |
| Cesarean delivery (%) | 13.1 | 10.8 |
| sTNFR1 (ng/mL) | 1.82 ± 0.44 | 1.91 ± 0.40 |
| STNFR2 (ng/mL) | 4.31 ± 1.68 | 4.43 ± 1.24 |
| sTNFR2/sTNFR1 | 2.39 ± 0.81 | 2.41 ± 0.77 |
| Leptin (ng/mL) | 24.5 ± 12.71 | 23.90 ± 12.07 |
| Log MBL | 2.86 ± 0.87 | 2.99 ± 0.86 |

Data are Means ± SD, except cesarean delivery;
BMI: Body mass index; SBP: systolic blood pressure; DBP: diastolic blood pressure.
Data for sTNFR1, sTNFR2, and leptin were analysed as log 10 of raw data for t-test.
*p < 0.05 vs control;
†p = 0.01 vs control;
‡p < 0.001 vs control

TABLE 2
Relationship between MBL polymorphism
and occurrence of GDM.

| | | | |
|---|---|---|---|
| HP | GD | P | |
| n (%) | n (%) | value | OR (95% CI) |

Genotype

| | | | | | | |
|---|---|---|---|---|---|---|
| MBL wild | 111 | (64.2) | 53 | (50.5) | | |
| type | | | | | | |
| MBL mutate | 62 | (35.8) | 52 | (49.5) | <0.05 | 1.76 (1.04-2.96) |

G54D

| | | | | | | |
|---|---|---|---|---|---|---|
| GG | 125 | (72.3) | 59 | (57.3) | | |
| AG | 43 | (24.9) | 42 | (40.8) | <0.05 | 1.96 (1.13-3.35)* |
| AA | 5 | (2.9) | 2 | (1.9) | | |

R52C

| | | | | | |
|---|---|---|---|---|---|
| CC | 153 | (88.4) | 94 | (91.3) | |
| CT | 20 | (11.6) | 9 | (8.7) | ns |

Abbreviations:
G: wild-type allele, A = codon 54 minority allele. OR: odds ratio. CI: confidence interval. ns: non significant
*Odds ratio GG vs AG + AA.

### Discussion and Results:

The results of our study show that the mutant MBL allele confers a greater susceptibility for developing GDM. In particular, for women bearing the G54D mutation (OR: 1.96; CI: 1.13-3.35). GDM patients who carried the mutations required insulin therapy more frequently (30.4 vs 65.0%, p<0.05) and had heavier infants (3087.5 g ±395.5 vs 3359.6 g ±520.3 g, P<0.01) than GDM women homozygous for the wild type allele. An inverse correlation in GDM patients between foetal birth weight and plasma MBL levels (−0.320; p=0.002) was found, remaining significant after adjustment for age, pregravid maternal weight and week of delivery. Insulin dose was correlated with sTNFR2/sTNFR1 (r:0. 624; p<0.001) and inversely correlated to sTNFR1 (r: −0.346; p<0.05). In conclusion, pregnant women bearing a mutant MBL allele, in particular, the G54D mutation, have a greater risk for developing GDM and having heavier infants.

### Example 2

### Materials and Methods

### Animals

Male Wistar Hanover rats (60-80 g) were purchased from Harlan Ibérica, Spain. They were housed in a controlled environment on a 12-hr light/12-hr dark cycle and fed a standard chow diet. Water and food were available ad libitum. All experiments were performed according to the criteria of the Animal Ethics Committee at Parc Cientific de Barcelona.

### In Vitro Muscle Incubation

On the experimental day, rats were anesthetized with pentobarbital sodium (60 mg/kg ip) and the soleus muscle was carefully dissected and placed in 1 mL of cold Krebs-Henseleit Hepes buffer (pH 7.4), supplemented with 5 mM glucose, until all the muscles were collected. Soleus muscles (30±1 mg) were preincubated for 20 min at 37° C. in 2 mL of Krebs-Henseleit Hepes buffer containing 2% bovine serum albumin (fatty acid free-BSA, Sigma, St. Louis, Mo.) and 5 mM glucose (incubation medium). The media were gassed continuously with 95% O2-5% CO2. The media was then removed and the muscles were incubated for 90 min at 37° C. in 1 mL of fresh medium with the following additions: None (baseline), insulin (100 nM), MBL (50 μg/mL human mannan-binding lectin; US Biologicals, Swampscott, Mass.) or insulin plus MBL.

### Glucose and Fatty Acid Oxidation

For glucose oxidation studies, 3 μCi/mL D-[U]-14C-Glucose (Amersham Biosciences) were added to the incubation medium. For fatty acid oxidation studies, the incubation medium contained 3 μCi/mL [1]-14C-Palmitic Acid (Amersham Biosciences), 100 μM Palmitic Acid (Sigma), 50 μM NaOH, 0.5% EtOH and 0.8 mg/mL BSA. The muscles were incubated for 60 min at 37° C. Gassing was terminated after the initial 15 min. The test tubes were hermetically closed with turn-over flange rubber stoppers (Saint-Gobain Verneret, France) with a center well that contained a piece of filter paper secured with a staple. At the end of the incubation, the medium was acidified with 0.3 mL of 0.5 N H₂SO₄ and the filter paper was saturated with 200 μL of benzetonium hydroxide (Hyamine; Sigma, St. Louis, Mo.) to trap gaseous 14CO₂ liberated after the acidification. The vials were shaken at 37° C. for 60 min, and the filter papers were removed and transferred to vials for liquid scintillation counting.

### Results:

Incubation of mouse soleus muscle with human MBL markedly increased fat oxidation. Under these conditions, glucose oxidation was not modified by exposure to MBL. In agreement with this MBL-induced increase in fat oxidation, plasma MBL-A concentration in mice circulated in proportion to the degree of insulin sensitivity. Thus, the concentrations of MBL-A were lower in insulin resistant obese ob/ob mice (n=9) compared to the control group (n=9)(20±4 and 26±3.7 ng/l, respectively, p<0.001).

### Discussion:

These findings imply that MBL increases fatty acid oxidation and that this is the mechanism through which lower risk of macrosomia can be obtained from MBL administration.

What is claimed is:

1. A kit for detecting a predisposition to gestational diabetes mellitus (GDM) in a woman, comprising appropriate means for detecting, in a sample taken from said woman, the presence or the absence of:
(i) at least one mutation, in exon 1 of the MBL2 gene, selected from the group consisting of a mutation that provokes the amino acid substitution R52C, a mutation that provokes the amino acid substitution G54D and a mutation that provokes the amino acid substitution G57E in the corresponding encoded polypeptide; and/or
(ii) the haplotype HY in the promoter region of the MBL2 gene.

2. The kit according to claim 1, wherein the woman is in the first trimester of pregnancy or is willing to get pregnant.

3. The kit according to claim 1, wherein the means includes PCR technology.

4. The kit according to claim 1, wherein the kit further comprises appropriate instructions explaining that:
(i) the presence of at least one mutation, in exon 1 of the MBL2 gene, selected from the group consisting of a mutation that provokes the amino acid substitution R52C, a mutation that provokes amino acid substitution G54D and a mutation that provokes amino acid substitution G57E in the corresponding encoded polypeptide; and/or
(ii) the absence of the haplotype HY in the promoter region of the MBL2 gene, results in an increased predisposition to have or develop gestational diabetes mellitus (GDM).

5. A method of use of a composition comprising at least one mannan-binding lectin (MBL) subunit, or at least one mannan-binding lectin (MBL) oligomer comprising at least one mannan-binding lectin (MBL) subunit, the method comprising administering a medicament for the preventive or therapeutic treatment of gestational diabetes mellitus (GDM) in a woman who one or the other or both:
(i) carries at least one mutation, in exon 1 of the MBL2 gene, selected from the group consisting of a mutation that provokes the amino acid substitution R52C, a mutation that provokes the amino acid substitution G54D and a mutation that provokes the amino acid substitution G57E in the corresponding encoded polypeptide; and/or
(ii) does not carry the haplotype HY in the promoter region of the MBL2 gene.

6. The method of claim 5, wherein the treatment is preventive and the woman is pregnant or is willing to get pregnant.

7. The method of claim 5, wherein the treatment is therapeutic and the woman suffers from gestational diabetes mellitus.

8. The method according to claim 5, wherein the woman carries a mutation that provokes the amino acid substitution G54D.

9. The method according to claim 5, wherein the MBL is recombinant human MBL.

10. The method according to claim 5, wherein the medicament comprises an injectable depot composition, which after introduction into the woman has a therapeutically release profile of MBL.
